(19) United States
(12) Patent Application Publication
(10) Pub. No.: US 2013/0085081 A1
(43) Pub. Date: Apr. 4, 2013

(54) RISK PREDICTION OF DEVELOPING DRUG-INDUCED LUNG INJURY AND DETECTION METHOD AND KIT OF GENE FOR RISK PREDICTION

(71) Applicants: Fumiaki Koizumi, Tokyo (JP); Shintaro Kanda, Tokyo (JP); Tomohide Tamura, Tokyo (JP); Koichi Goto, Chiba (JP); Masaru Sekijima, Tokyo (JP); Akira Ohide, Tokyo (JP); Kazuto Nishio, Osaka (JP)

(72) Inventors: Fumiaki Koizumi, Tokyo (JP); Shintaro Kanda, Tokyo (JP); Tomohide Tamura, Tokyo (JP); Koichi Goto, Chiba (JP); Masaru Sekijima, Tokyo (JP); Akira Ohide, Tokyo (JP); Kazuto Nishio, Osaka (JP)

(73) Assignees: National Cancer Center, Tokyo (JP); Kinki University, Osaka (JP); Mitsubishi Chemical Medience Corporation, Tokyo (JP)

(21) Appl. No.: 13/702,574

(22) PCT Filed: Jun. 8, 2011
(86) PCT No.: PCT/JP2011/063142

§ 371 Date: Dec. 6, 2012

(30) Foreign Application Priority Data
Jun. 8, 2010 (JP) 2010-130992

(51) Int. Cl. C12Q 1/68

(52) U.S. Cl. CPC C12Q 1/6813
USPC 506/9; 435/6.11; 536/24.31; 536/24.33

(57) ABSTRACT

Disclosed are a method of detecting the presence or absence of a single nucleotide polymorphism of a gene, for prediction of the risk of developing drug-induced lung injury, or for improving a therapeutic method, and a kit for carrying out the detection method. The detection method is characterized by comparing an ABCB1 gene in a biological sample with a wild-type ABCB1 gene to detect the presence or absence of a single nucleotide polymorphism in the ABCB1 gene in the biological sample, in particular, by determining the nucleotide at position 3751 of the CDS of the ABCB1 gene. The kit comprises an oligonucleotide probe which specifically binds to a single nucleotide polymorphism in an ABCB1 gene under selective binding conditions, or an oligonucleotide primer which amplifies a nucleic acid sequence comprising a single nucleotide polymorphism in an ABCB1 gene.

### TECHNICAL FIELD

The present invention relates to a method of detecting the presence or absence of a single nucleotide polymorphism of an ABCB1 gene, for prediction of the risk of developing drug-induced lung injury (in particular, interstitial lung disease) with high fatality rate and severity, or for improving a therapeutic method, and a kit for carrying out the detection method. Further, the present invention relates to the method of detecting the presence or absence of a single nucleotide polymorphism, wherein the single nucleotide polymorphism is single nucleotide polymorphism rs28364274 of the ABCB1 gene, which is a substitution of adenine for guanine at position 4169 of the gene of SEQ ID NO: 1, and a kit for carrying out the detection method.

### BACKGROUND ART

Epidermal growth factor receptor tyrosine kinase inhibitors (EGFR-TKIs), such as gefitinib (Iressa (registered trademark)) or erlotinib (Tarceva (registered trademark)), are therapeutic agents for non-small cell lung cancer, and it is recognized that their role is large, particularly, in non-small cell lung cancer of the Japanese with relatively high frequency of having activating EGFR gene mutations as a factor of sensitivity, and the stage and situation for which they are indicated is expanding (non-patent literatures 3-5).

The most important point to be noted during the administration of an EGFR tyrosine kinase inhibitor is interstitial lung disease (ILD) as an adverse event (non-patent literatures 6-8). The incidence of ILD was seldom an issue in races other than the Japanese, but was relatively high in the Japanese, and therefore, for a certain time it became a social problem due to a specific acute onset and high fatality rate.

In a cohort study of ILD caused by anti-cancer drugs in Japanese patients with lung caner, whereas the incidence of ILD caused by general anti-cancer drugs was 2.1%, the incidence of ILD caused by gefitinib was 4.0%, the fatality rates were 27.9% and 31.6%, respectively, and the prognosis was poor. Clinical risk factors include, for example, history of ILD or lung fibrosis, history of smoking, male, elderly, and complications of heart disease (non-patent literatures 9-11), and the administration to a patient having a lot of risk factors can be avoided in the medical field. However, some patients having no risk factors as above have developed ILD, and it has not been possible to completely avoid the onset of ILD using clinical findings alone.

Under these circumstances, it is desired to enable prediction of the risk of developing ILD for a patient in which a therapy with EGFR tyrosine kinase inhibitors is considered. Further, it is also desired to improve the treatment of that adverse event with a high fatality rate and to improve the safety of treatment with anti-cancer drugs including EGFR tyrosine kinase inhibitors.

Aside from this, it is known that diarrhea caused by gefitinib administration is highly associated with a single nucleotide polymorphism in an ABCG2 gene, but is not associated with single nucleotide polymorphisms in an ABCB1 gene and an EGFR gene (non-patent literature 12). Further, it is known that no single nucleotide polymorphisms in these genes are associated with skin toxicity. However, these single nucleotide polymorphisms cannot be used as an index for the onset of drug-induced lung injury.

### CITATION LIST

### Non-Patent Literature

[Non-patent literature 1] Lynch T J, Bell D W, Sordellaet R, et al. Activating mutations in the epidermal growth factor receptor underlying responsiveness of non-small-cell lung cancer to gefitinib. N Engl J Med 350: 2129-2139, 2004.

[Non-patent literature 2] Paez J G, Janne P A, Lee J C, et al. EGFR mutations in lung cancer: correlation with clinical response to gefitinib therapy. Science 304: 1497-1500, 2004.

[Non-patent literature 3] Kim E S, Hirsh V, Mok T, et al. Gefitinib versus docetaxel in previously treated non-small-cell lung cancer (INTEREST): a randomised phase III trial. Lancet 372: 1809-1818, 2008.

[Non-patent literature 4] Shepherd F A, Rodrigues P J, Ciuleanu T, et al. Erlotinib in previously treated non-small-cell lung cancer. N Engl J Med 353: 123-132, 2005.

[Non-patent literature 5] Mok T S, Wu Y L, Thongprasert S, et al. Gefitinib or carboplatin-paclitaxel in pulmonary adenocarcinoma. N Engl J Med 361: 947-957, 2009.

[Non-patent literature 6] Inoue A, Saijo Y, Maemondo M, et al. Severe acute interstitial pneumonia and gefitinib. Lancet 361: 137-139, 2003

[Non-patent literature 7] Okamoto I, Fujii K, Matsumoto M, et al. Diffuse alveolar damage after ZD1839 therapy in a patient with non-small cell lung cancer. Lung Cancer 40: 339-342, 2003

[Non-patent literature 8] Ieki R, Saitoh E, Shibuya M. Acute lung injury as a possible adverse drug reaction related to gefitinib. Eur Respir J 22, 179-181

[Non-patent literature 9] Kudoh S, Kato H, Nishiwaki Y, et al. Interstitial lung disease in Japanese patients with lung cancer: a cohort and nested case-control study. Am J Respir Crit Care Med 177:1348-1357, 2008

[Non-patent literature 10] Ando M, Okamoto I, Yamamoto N, et al. Predictive factors for interstitial lung disease, antitumor response, and survival in non-small-cell lung cancer patients treated with gefitinib. J Clin Oncol 24: 2549-2556, 2006

[Non-patent literature 11] Takano T, Ohe Y, Kusumoto M, Tateishi U, et al. Risk factors for interstitial lung disease and predictive factors for tumor response in patients with advanced non-small cell lung cancer treated with gefitinib. Lung Cancer 45: 93-104, 2004

[Non-patent literature 12] Cusatis G, Gregorc V, Li J, Spreafico A, et al. Pharmacogenetics of ABCG2 and adverse reactions to gefitinib. J Natl Cancer Inst 98(23):1739-1742, 2006.

### SUMMARY OF INVENTION

### Technical Problem

For the risk prediction and improvement of therapy described above, the present inventors considered it necessary to find polymorphisms in the genes involved in the onset of drug-induced lung injury, and to elucidate the mechanisms of developing drug-induced lung injury by functional analysis of the single nucleotide polymorphisms.

An object of the present invention is to establish a method of detecting the presence or absence of a single nucleotide polymorphism of a gene, for prediction of the risk of developing drug-induced lung injury, or for improving a therapeutic method, by finding polymorphisms in the genes involved in the onset of drug-induced lung injury including interstitial lung disease (ILD), and by elucidating the mechanisms of developing drug-induced lung injury by functional analysis of the single nucleotide polymorphisms.

### Solution to Problem

The present inventors conducted intensive study to solve the object, and found a single nucleotide polymorphism in a gene involved in the onset of drug-induced lung injury, and elucidated part of the mechanisms of developing drug-induced lung injury by functional analysis of the single nucleotide polymorphism.

As described in detail in the Examples below, there are persons who develop acute lung injury, by the administration of EGFR tyrosine kinase inhibitors (patient group), and persons who do not develop acute lung injury (control group), depending on the presence or absence of single nucleotide polymorphisms in each individual. The present inventors elucidated a single nucleotide polymorphism different between both groups, and found that, with respect to single nucleotide polymorphism rs28364274 of an ABCB1 (ATP-binding cassette transporter B1) gene, 4 (33%) of 12 cases had an A allele in the patient group, whereas no case with the A allele was detected in the control group. Further, since the A allele of single nucleotide polymorphism rs28364274 is extremely rare in some non-Japanese races, it was considered that the A allele of single nucleotide polymorphism rs28364274 detected in the 4 cases was extremely specific, and it was found that this result did not contradict the fact that ILD is often developed in the Japanese.

As described above, the present inventors found that the presence or absence of the single nucleotide polymorphism in the ABCB1 gene was involved in the onset of drug-induced lung injury, and completed the present invention.

The present invention provides:

[1] A method of predicting the risk of developing drug-induced lung injury, characterized by determining the nucleotide at position 3751 of the CDS of an ABCB1 gene (the nucleotide at position 4169 of the nucleotide sequence of SEQ ID NO: 1).

[2] The method of [1], wherein it is judged that the risk of developing drug-induced lung injury is high, when the nucleotide is adenine.

[3] A method of detecting the presence or absence of a single nucleotide polymorphism in an ABCB1 gene in a biological sample, for prediction of the risk of developing drug-induced lung injury, by comparing the ABCB1 gene in the biological sample with a wild-type ABCB1 gene.

[4] The method of [3], wherein the single nucleotide polymorphism is a substitution of adenine for guanine in the nucleotide at position 3751 of the CDS of an ABCB1 gene (the nucleotide at position 4169 of the nucleotide sequence of SEQ ID NO: 1).

[5] A method of any one of [1] to [4], comprising the steps of:

(a) isolating a DNA comprising a nucleotide site with a single nucleotide polymorphism in an ABCB1 gene from a nucleic acid sample prepared from a subject,

(b) bringing the DNA sample isolated in (a) into contact with a substrate immobilized with an ABCB1 gene fragment comprising the nucleotide site with the single nucleotide polymorphism, as a nucleotide probe, under selective binding conditions, and

(c) detecting the intensity of hybridization of the DNA sample with the nucleotide probe immobilized on the substrate.

[6] A method of detecting the presence or absence of a single nucleotide polymorphism of [3] or [4], comprising the steps of:

(a) isolating a DNA comprising a single nucleotide polymorphism in an ABCB1 gene from a nucleic acid sample prepared from a subject,

(b) determining the nucleotide sequence of the isolated DNA sample, and

(c) comparing the DNA nucleotide sequence determined in (b) with a control.

[7] The method of detecting the presence or absence of a single nucleotide polymorphism of [3] or [4], by detecting the single nucleotide polymorphism in the ABCB1 gene, using an antibody, as a mutation of an amino acid sequence resulting from the single nucleotide polymorphism in the ABCB1 gene.

[8] The method of [1] or [2], by detecting the nucleotide at position 3751 of the CDS of the ABCB1 gene, using an antibody, as a mutation of an amino acid sequence resulting from a single nucleotide polymorphism in the nucleotide.

[9] An oligonucleotide probe for predicting the risk of developing drug-induced lung injury, which specifically binds to a single nucleotide polymorphism in an ABCB1 gene under selective binding conditions.

[10] An oligonucleotide primer for predicting the risk of developing drug-induced lung injury, which amplifies a nucleic acid sequence comprising a single nucleotide polymorphism in an ABCB1 gene.

[11] A kit for predicting the risk of developing drug-induced lung injury, comprising the oligonucleotide probe of [9], wherein the presence or absence of the single nucleotide polymorphism in the ABCB1 gene in a biological sample is detected by comparing the ABCB1 gene in the biological sample with a wild-type ABCB1 gene.

As the “ABCB1 (ATP-binding cassette transporter B1) gene”, the nucleotide sequence (mRNA) of the wild-type ABCB1 gene is registered as NCBI number NM 000927.3 (nucleotide sequence of SEQ ID NO: 1; total number of nucleotides=4872). The CDS (coding region from the initiation codon to the stop codon) is the sequence consisting of nucleotides 419-4261 in SEQ ID NO: 1.

As the “ABCB1 protein” encoded by the “ABCB1 gene”, the amino acid sequence of the wild-type ABCB1 protein is registered as NCBI number NP_{—}000918.2 (amino acid sequence of SEQ ID NO: 2; total number of amino acids=1280).

The “single nucleotide polymorphism of the ABCB1 gene” utilized in the present invention is a single nucleotide polymorphism in the nucleotide at position 3751 of the CDS of the ABCB1 gene (the nucleotide at position 4169 of the nucleotide sequence of SEQ ID NO: 1), and a substitution of adenine (A) for guanine (G) in the wild-type. This single nucleotide substitution is registered as NCBI number rs28364274 (nucleotide sequence of SEQ ID NO: 3; nucleotide R (i.e., G or A) at position 256 of SEQ ID NO: 3 indicates the single nucleotide substitution).

The term “genotype” as used herein refers to the particular allelic form of a gene, which can be defined by the particular nucleotide(s) present at a particular site(s) in a nucleic acid sequence.

The terms “single nucleotide polymorphism”, “allele”, or “SNP” refer to one specific form of a gene in a population, the specific form differing from other forms of the same gene in the sequence of at least one, and frequently more than one, variant sites within the sequence of the gene. The sequences at these variant sites that differ between different alleles of the gene are termed “gene sequence variances” or “variances” or “variants”. Other terms known in the art to be equivalent include mutation and polymorphism, although mutation is often used to refer to an allele associated with a deleterious phenotype.

An individual in which both alleles are the same is referred to as a homozygote, an individual in which both alleles are different is referred to as a heterozygote.

For example, when the alleles are A (adenine) or G (guanine), the genotypes include three types, i.e., (AA), (AG), and (GG).

The term “probe” refers to a molecule which can detectably distinguish between target molecules differing in structure. Detection can be accomplished in a variety of different ways depending on the type of probe used and the type of target molecule. Thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and preferably nucleic acid hybridization probes.

The term “primer” as used herein refers to an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature. A primer must be sufficiently long to prime the synthesis of extension products in the presence of an agent for polymerase. A typical primer contains at least about 5 nucleotides in length of a sequence substantially complementary to the target sequence, but somewhat longer primers are preferred. Usually primers contain about 15 to 26 nucleotides, but longer primers may also be employed.

A primer will always contain a sequence substantially complementary to the target sequence, that is the specific sequence to be amplified, to which it can anneal. A primer may, optionally, also comprise a promoter sequence. The term “promoter sequence” defines a single strand of a nucleic acid sequence that is specifically recognized by an RNA polymerase that binds to a recognized sequence and initiates the process of transcription by which an RNA transcript is produced. In principle, any promoter sequence may be employed for which there is a known and available polymerase that is capable of recognizing the initiation sequence. Known and useful promoters are those that are recognized by certain bacteriophage polymerases, such as bacteriophage T3, T7 or SP6.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least 50/cm², more preferably at least about 100/cm², even more preferably at least about 500/cm², and still more preferably at least 1,000/cm². A DNA microarray as used herein is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes, and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases.

The term “antibody” is meant to be an immunoglobulin protein that is capable of binding an antigen. Antibody as used herein is meant to include antibody fragments, such as F(ab′)₂, Fab′, and Fab, capable of binding the antigen or antigenic fragment of interest.

Nucleic acid molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Rolff, A et al. PCR: Clinical Diagnostics and Research, Springer (1994)).

### Advantageous Effects of Invention

According to the present invention, a method of detecting the presence or absence of a single nucleotide polymorphism of an ABCB1 gene in a biological sample, for prediction of the risk of developing drug-induced lung injury (in particular, interstitial lung disease) with high fatality rate and severity, or for improving a therapeutic method, and a kit for carrying out the detection method, are provided.

### BRIEF DESCRIPTION OF THE DRAWINGS

FIG. 1 is a graph showing the percentages of each gene group into which 768 SNPs used in Example 1 are classified.

FIG. 2 is a sequence alignment showing the result of the comparison between the nucleotide sequences of single nucleotide polymorphism rs28364274 and wild-type ABCB1 gene (NM_{—}000927.3).

### DESCRIPTION OF EMBODIMENTS

The present invention provides a method of detecting the presence or absence of a single nucleotide polymorphism in the ABCB1 gene in a biological sample, by comparing it with the wild-type ABCB1 gene. Further the present invention provides a method of detecting the presence or absence of a single nucleotide polymorphism, wherein the single nucleotide polymorphism is a substitution of adenine for guanine in the nucleotide at position 3751 of the CDS of the ABCB1 gene (the nucleotide at position 4169 of the nucleotide sequence of SEQ ID NO: 1).

Furthermore, the present invention provides a novel method of determining the possibility of developing drug-induced lung injury, in a patient with the risk of developing cancer, or in a therapy of targeting the epidermal growth factor receptor (EGFR) for a human patient suffering from cancer. The method includes the step of determining whether or not the patient has the presence or absence of a single nucleotide polymorphism in the ABCB1 gene. The presence of such a different indicates that the risk of developing drug-induced lung injury is high. In some cases where such a polymorphism is present, tyrosine kinase inhibitors are not administered to patients.

The tyrosine kinase inhibitor administered to an identified patient as above may be, for example, an anilinoquinazoline or an irreversible tyrosine kinase inhibitor, such as EKB-569, HKI-272 and/or HKI-357 (Wyeth). Preferably, the anilinoquinazoline is a synthetic anilinoquinazoline, and most preferably the synthetic anilinoquinazoline is gefitinib and erlotinib.

The lung injuries of interest in the present invention are diseases caused by an inflammatory reaction in the lung interstitium (partition walls between the alveoli) as the primary lesion. Examples of such diseases include interstitial lung disease (ILD), preferably drug-induced lung injuries caused by the administration of a drug. For example, drug-induced lung injuries caused by tyrosine kinase inhibitors such as gefitinib or erlotinib, which have characteristic onset style, imaging findings, and severity (in particular, high severity) in common, are most preferable.

Further, a tyrosine kinase inhibitor (TKI) therapy in the therapy of targeting the epidermal growth factor receptor (EGFR) is not effective in the majority of patients suffering from epithelial cell cancers, such as lung cancer, ovarian cancer, breast cancer, brain cancer, colon cancer, and prostate cancer, as described in JP 2007-531525. Since the presence of somatic mutation in the kinase domain of EGFR results in a substantial increase in the sensitivity of EGFR to the TKI therapy with gefitinib or erlotinib, there is a high possibility that patients with this mutation respond to the current TKI therapy, such as gefitinib. Therefore, it is disclosed that, by detecting the presence or absence of at least one gene mutation in the kinase domain of an erbB1 gene in the patients, in comparison with the wild-type erbB1 gene, there is a high possibility that the EGFR targeting therapy is effective in patients having at least one difference.

In addition of detecting whether or not a patient has the presence or absence of the gene mutation of the ABCB1 gene in the present invention, the presence or absence of at least one gene mutation in the kinase domain of the erbB1 gene in the patient can be detected, in comparison with the wild-type erbB1 gene, to use it as an index for determining the administration of a tyrosine kinase inhibitor to the patient.

In the present invention, the ABCB1 gene may be obtained from a biological sample collected from a human suffering from cancer or having the risk of developing cancer, preferably a biological sample collected from a patient in which a therapy with EGFR tyrosine kinase inhibitors is considered.

The “biological sample” may be any sample containing somatic cells, and normal cells or tumor cells may be used. A sample of tissue or fluid isolated from an individual, including but not limited to, for example, blood, tumor biopsy, spinal fluid, pleural fluid, nipple aspirates, lymph fluid, the external sections of the skin, oral mucosal tissue, respiratory, intestinal, and genital tracts, saliva, hair, nails, milk, cells (including but not limited to blood cells), tumors, organs, and also samples of in vitro cell culture constituent. Samples may be either paraffin-embedded or frozen tissue.

Non-invasive peripheral blood mononuclear cells from whole blood, or invasive tumor tissues by biopsy, which can be easily used, are preferable.

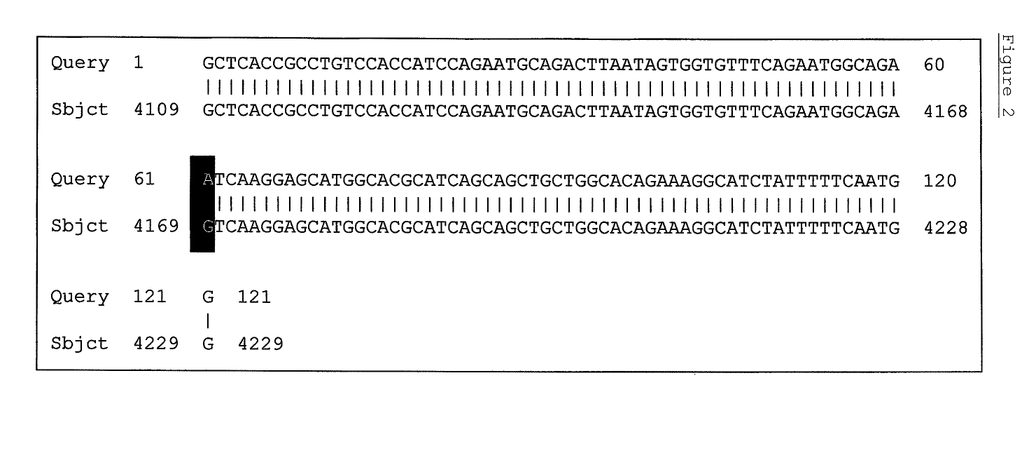

In the present invention, the presence or absence of the single nucleotide polymorphism in single nucleotide polymorphism rs28364274 of the ABCB1 gene is detected using the substitution of A (adenine) for G (guanine) at position 4169 of the wild-type ABCB1 gene (NM_{—}000927.3) as an index. The result of the comparison between the nucleotide sequences of single nucleotide polymorphism rs28364274 and wild-type ABCB1 gene (NM_{—}000927.3) is shown in FIG. 2. In FIG. 2, the sequence “query” (1 to 121) is a partial sequence (SEQ ID NO: 4) of the nucleotide sequence (SEQ ID NO: 3) of single nucleotide polymorphism rs28364274, and the sequence “sbjct” (4109 to 4229) is a partial sequence of the wild-type ABCB1 gene (NM 000927.3; SEQ ID NO: 1). The white characters on black background show the portion of the single nucleotide polymorphism. This substitution results in an amino acid substitution of I (isoleucine) for V (valine) at position 1251 of the wild-type ABCB1 protein (NP_{—}000918.2).

Therefore, the detection of the presence or absence of the single nucleotide polymorphism can be achieved by either or both of a detection of nucleic acid mutation on the alleles or a detection of amino acid mutation on the alleles.

A large number of known analytical procedures may be used to detect the presence or absence of the single nucleotide polymorphism in the ABCB1 gene of the present invention. In general, the detection of the presence or absence of single nucleotide polymorphism requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of single nucleotide polymorphisms are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

Protein mutation detection methods listed in Table 4 may also be used.

TABLE 1
Mutation Detection Techniques

General:
DNA sequencing
Sequencing by hybridization
Scanning:
PTT (Protein truncation test)(*)
SSCP (Single-strand conformation polymorphism analysis)
DGGE (Denaturing gradient gel electrophoresis)
TGGE (Temperature gradient gel electrophoresis)
Cleavase
Heteroduplex analysis
CMC (Chemical mismatch cleavage)
Enzymatic mismatch cleavage
Hybridization Based
Solid phase hybridization:
Dot blots
MASDA
Reverse dot blots
Oligonucleotide arrays (DNA Chips)
Solution phase hybridization:
Taqman (TM) - U.S. Pat. No. 5,210,015 & U.S. Pat. No. 5,487,972
(Hoffmann-La Roche) Molecular Beacons - Tyagi et al (1996), Nature
Biotechnology, 14, 303; WO 95/13399 (Public Health Inst., New York)
PNA-LNA PCR clamp - JP Patent 4216266
Cycleave
Extension Based:
ARMS (TM)(Amplification refractory mutation system)
ALEX (TM)(Amplification refractory mutation system linear
extension) - European Patent No. EP 332435 B1 (Zeneca Limited)
COPS (Competitive oligonucleotide priming system) - Gibbs et al
(1989), Nucleic Acids Research, 17, 2347.
Incorporation Based:
Mini-sequencing
APEX (Arrayed primer extension)
Restriction Enzyme Based:
RFLP (Restriction fragment length polymorphism)
Restriction site generating PCR
Ligation Based:
OLA (Oligonucleotide ligation assay)
Other:
Invader assay
(Note:
not useful for detection of promoter polymorphisms.)

TABLE 2
Signal Generation or Detection Systems

Fluorescence:
FRET (Fluorescence resonance energy transfer)
Fluorescence quenching
Fluorescence polarization - United Kingdom Patent No. 2228998
(Zeneca Limited)
Other:
Chemiluminescence
Electrochemiluminescence
Raman
Radioactivity
Colorimetric
Hybridization protection assay
Mass spectrometry

TABLE 3
Further Amplification Methods

SSR (Self sustained replication)
NASBA (Nucleic acid sequence based amplification)
LCR (Ligase chain reaction)
SDA (Strand displacement amplification)
b-DNA (Branched DNA)

TABLE 4
Protein Mutation Detection Methods

Immunoassay
Immunohistology
Peptide sequencing

Preferred mutation detection techniques include Oligonucleotide arrays (DNA Chips), DNA sequencing, ARMS™, ALEX™, COPS, Taqman, PNA-LNA PCR clamp, Molecular Beacons, RFLP, and restriction site based PCR and FRET techniques.

Particularly preferred methods include Oligonucleotide arrays (DNA Chips), DNA sequencing, Taqman, and PNA-LNA PCR clamp.

Hereinafter some detection methods will be exemplified.

In a detection method, the detection of the single nucleotide polymorphism can be carried out, for example, by directly sequencing the nucleotide sequence of the ABCB1 gene from a subject. In this method, a DNA sample is prepared from a biological sample obtained from a subject. These samples may be prepared from chromosomal DNA, or RNA extracted from a tissue or cells.

In this method, a DNA containing the single nucleotide polymorphism on the ABCB1 gene is isolated. This isolation can also be carried out by PCR or the like using chromosomal DNA, or RNA as a template, together with primers which hybridize to nucleic acid sequences capable of amplifying a nucleic acid sequence containing the single nucleotide polymorphism on the ABCB1 gene. In this method, the nucleotide sequence of the isolated DNA is determined. The determination of the nucleotide sequence of the isolated DNA can be carried out by a method known to those skilled in the art.

In this method, the nucleotide sequence determined of the DNA is compared with that of a control. The control as used herein means the sequence of the normal (wild-type) ABCB1 gene. In general, the sequence (NM 000927.3) of the ABCB1 gene registered as the wild-type in the GenBank may be used.

In the detection method of the present invention, various methods capable of detecting polymorphism can be used other than the method directly determining the nucleotide sequence of a DNA, which was derived from the subject, as described above. For example, the following methods may be used. In an embodiment, a DNA sample is first prepared from a subject, and is digested with restriction enzymes. Then, the DNA fragments are separated in accordance with their size, and the detected sizes of the DNA fragments are compared with those of a control. In another embodiment, a DNA sample is first prepared from a subject. Then, a DNA containing the ABCB1 gene is amplified from the sample, and the amplified DNA is digested with restriction enzymes. After separating the DNA fragments according to their size, the detected sizes of the DNA fragments are compared with those of a control.

Such methods include, for example, a method utilizing Restriction Fragment Length Polymorphism (RFLP), PCR-RFLP, and the like. Specifically, when a mutation exists in the recognition sites of a restriction enzyme, or when a nucleotide insertion or deletion exists in a DNA fragment generated by a restriction enzyme treatment, the sizes of fragments that are generated after the restriction enzyme treatment vary in comparison with those of a control. The portion containing the mutation is amplified by PCR, and then, is treated with appropriate restriction enzymes to detect these mutations as a difference of the mobility of bands after electrophoresis. Alternatively, the presence or absence of a mutation can be detected by carrying out Southern blotting with a probe DNA of the present invention after treating chromosomal DNA with restriction enzymes followed by electrophoresis. The restriction enzymes to be used can be appropriately selected in accordance with each mutation. The Southern blotting can be conducted not only on the genomic DNA but also on cDNAs directly digested with restriction enzymes, wherein the cDNAs are converted by the use of a reverse transcriptase from RNAs prepared from a subject. Alternatively, after amplifying a DNA containing the ABCB1 gene by PCR using the cDNA as a template, the amplified DNA may be digested with restriction enzymes, and the difference of mobility may be examined.

In still another embodiment, a DNA sample is first prepared from a subject. Then, a DNA containing the ABCB1 gene is amplified. Thereafter, the amplified DNA is dissociated into single strand DNAs, and the resulting single strand DNAs are separated from each other on a non-denaturing gel. The mobility of the separated single strand DNAs on the gel is compared with that of a control.

Such methods include, for example, PCR-SSCP (single-strand conformation polymorphism) (“Cloning and polymerase chain reaction-single-strand conformation polymorphism analysis of anonymous Alu repeats on chromosome 11”. Genomics. 1992 Jan 1; 12(1): 139-146.; “Detection of p53 gene mutations in human brain tumors by single-strand conformation polymorphism analysis of polymerase chain reaction products.” Oncogene. 1991 Aug 1; 6(8): 1313-1318.; “Multiple fluorescence-based PCR-SSCP analysis with postlabeling.”, PCR Methods Appl. 1995 Apr 1; 4(5): 275-282). This method is particularly preferable for screening many DNA samples, since it has advantages such as: comparative simplicity of operation; small amount of a test sample required; and so on. The principle of the method is as follows. Each single strand DNA dissociated from a double-strand DNA fragment forms a unique higher conformation depending on its nucleotide sequence. When the dissociated DNA chains are electrophoresed on a polyacrylamide gel without a denaturant, complementary single-stranded DNAs having the same chain length shift to different positions in accordance with the difference of the higher conformations. The higher conformation of a single-stranded DNA changes even by a substitution, deletion, or insertion of one base, and the change results in a different mobility by polyacrylamide gel electrophoresis. Accordingly, the presence of a mutation in a DNA fragment due to point mutation, deletion, insertion, or the like can be detected by detecting the change of the mobility.

More specifically, a DNA containing the ABCB1 gene is first amplified by PCR or the like. Preferably, a DNA of a length of about 200 by to 400 by is amplified. Those skilled in the art can appropriately select the reaction conditions and such for the PCR. DNA products amplified by PCR can be labeled by primers which are labeled with isotopes such as ³²P, fluorescent dyes, biotin, or the like. Alternatively, the amplified DNA products can also be labeled by conducting PCR in a PCR reaction solution containing substrate nucleotides which are labeled with isotopes such as ³²P, fluorescent dyes, biotin, or the like. Further, the labeling can also be carried out by adding substrate nucleotides which are labeled with isotope such as ³²P, fluorescent dyes, biotin, or the like, to the amplified DNA fragment using Klenow enzyme or the like, after the PCR reaction. Then, the obtained labeled DNA fragment is denatured by heating or the like, and electrophoresis is carried out on a polyacrylamide gel without a denaturant such as urea. The conditions for the separation of the DNA fragment by this electrophoresis can be improved by adding an appropriate amount (about 5% to 10%) of glycerol to the polyacrylamide gel. Further, although the conditions for electrophoresis vary depending on the property of each DNA fragment, it is usually carried out at room temperature (20 to 25° C.). When a preferable separation is not achieved at this temperature, a temperature at which optimum mobility can be achieved is examined from 4 to 30° C. The mobility of the DNA fragment after the electrophoresis is detected by autoradiography with X-ray films, scanner for detecting fluorescence, or the like, to analyze the result. When a band with different mobility is detected, the presence of a mutation can be confirmed by directly excising the band from the gel, amplifying it again by PCR, and directly sequencing the amplified fragment. Further, the bands can also be detected by staining the gel after electrophoresis with ethidium bromide, silver staining, or the like, without using labeled DNAs.

In still another method, a DNA sample is first prepared from a subject. A DNA containing the ABCB1 gene is amplified, and then, the amplified DNA is separated on a gel with gradient concentration of a DNA denaturant. The mobility of the separated DNAs on the gel is compared with that of a control.

For example, denaturant gradient gel electrophoresis (DGGE) and the like can be exemplified as such a method. The DGGE comprises the steps of electrophoresing a mixture of DNA fragments on a polyacrylamide gel with gradient concentration of denaturant, and separating the DNA fragment of interest in accordance with the difference of instability of each fragment. When an unstable DNA fragment containing a mismatch(es) reaches a region with a certain concentration of the denaturant in the gel, a DNA sequence near the mismatch is partially dissociated to single strands because of its instability. The mobility of the partially-dissociated DNA fragment becomes remarkably slow, and it can be separated from a completely double-stranded DNA without such a dissociated portion, based on the difference in mobility. Specifically, a DNA containing the ABCB1 gene is amplified by PCR or the like with primers of the present invention or the like; the amplified DNA fragment is electrophoresed on a polyacrylamide gel with gradient concentration of denaturant such as urea; and the result is compared with that of a control. The presence or absence of a mutation can be detected by detecting the difference in mobility, because when a mutation is present in the DNA fragment, the DNA fragment converts into single-strands at a portion with a lower concentration of denaturant, and the moving speed becomes remarkably slow.

In still another method, a DNA containing an ABCB1 gene prepared by a subject, and a substrate on which a nucleotide probe which hybridizes to the DNA are immobilized are prepared. The DNA is brought into contact with the substrate. The single nucleotide polymorphism of the ABCB1 gene is detected by detecting the DNA which hybridizes to the nucleotide probe immobilized on the substrate.

As such a method, a DNA array method (SNP ichi-enki-takei no senryaku (Strategy of single nucleotide polymorphism SNP), Kenichi Matsubara and Yoshiyuki Sakaki, Nakayama shoten, p. 128-135, 2000) may be exemplified. A DNA sample containing an ABCB1 gene may be prepared from a subject by a method well-known to those skilled in the art. In a preferred embodiment for preparing the DNA sample, it may be prepared from chromosomal DNA, or RNA, extracted from a tissue or cells. For the preparation of the DNA sample from chromosomal DNA in this method, for example, a DNA containing the ABCB1 gene may be prepared by PCR or the like using the chromosomal DNA as a template, together with primers which hybridize to the DNA containing the ABCB1 gene. A label for detection may be added to the resulting DNA sample by a method well-known to those skilled in the art, if necessary.

The term “substrate” as used herein refers to a board type material on which nucleotides can be immobilized. The term “nucleotides” as used herein includes an oligonucleotide and a polynucleotide. The substrate used in the present invention is not limited so long as nucleotides can be immobilized thereon, but a substrate that is generally used in DNA array technique is preferred.

In general, a DNA array comprises thousands of nucleotides which are printed on the substrate at a high density. Usually, these DNAs are printed on the surface layer of a non-porous substrate. The surface layer of the substrate is usually glass, but a porous film, such as nitrocellulose membrane, can also be used.

As a method of fixation (array) of nucleotides, an array based on nucleotides developed by Affymetrix may be exemplified in the present invention. Oligonucleotides are usually synthesized in situ for the array of oligonucleotides. For example, in situ synthesis methods of oligonucleotides, such as photolithographic technique (Affymetrix) and ink-jet technique (Rosetta Inpharmatics) for fixing a chemical substance, are already known in the art, and any of these techniques can be used for the production of the substrate used in the present invention.

The nucleotide probe immobilized on the substrate is not limited, so long as it can be used to detect the single nucleotide polymorphism of the ABCB1 gene. Most preferably, a nucleotide probe which detects the single nucleotide polymorphism in the nucleotide at position 3751 of the CDS of the ABCB1 gene (the nucleotide at position 4169 of the nucleotide sequence of SEQ ID NO: 1), i.e., the substitution of adenine for guanine, is used. The probe is a probe which specifically hybridizes to, for example, the wild-type ABCB1 gene, or an ABCB1 gene with the single nucleotide polymorphism. So long as the specific hybridization can be achieved, it is not necessary that the nucleotide probe is completely complementary to a DNA containing the ABCB1 gene to be detected, or an ABCB1 gene with the single nucleotide polymorphism.

The length of the nucleotide probe which is immobilized on the substrate in the present invention is, generally 10 to 100 bases, preferably 10 to 50 bases, and more preferably 15 to 25 bases, when an oligonucleotide is immobilized.

Next, the cDNA sample is brought into contact with the substrate in the present invention. In this step, the DNA sample is hybridized to the nucleotide probe. The reaction liquid and the reaction conditions for the hybridization vary depending on various factors such as the length of the nucleotide probe immobilized on the substrate, but the hybridization may be generally carried out in accordance with a method well-known to those skilled in the art.

Next, the presence or absence, or the intensity of the hybridization between the DNA sample and the immobilized nucleotide probe is detected in the present invention. This detection may be carried out, for example, by analyzing a fluorescent signal using a scanner or the like. In DNA arrays, the DNA immobilized on the slide glass is generally designated “probe”, and the labeled DNA in the solution is designated “target”. Therefore, the nucleotide immobilized on the substrate is referred to as “nucleotide probe” in the present specification.

In addition to the above-mentioned methods, Allele Specific Oligonucleotide (ASO) hybridization can be used to detect only a mutation of single nucleotide substitution at a specific site. An oligonucleotide with a nucleotide sequence suspected of having a mutation is prepared, and is subjected to hybridization with a DNA sample. When a mutation containing single nucleotide substitution is present, the efficiency of hybridization is reduced. The reduction can be detected by, for example, Southern blotting, or a method utilizing a specific fluorescent reagent that has a characteristic to quench by intercalation into the gap of the hybrid. Further, the detection may be also conducted by ribonuclease A mismatch truncation. Specifically, a DNA containing the ABCB1 gene is amplified by PCR or the like, and the amplified DNA is hybridized with a labeled RNA which is prepared from an ABCB1 cDNA or the like incorporated into a plasmid vector or the like. The presence of a mutation can be detected with autoradiography or the like, after cleaving a site(s) that forms a single-stranded conformation, due to the existence of the mutation containing single nucleotide substitution, with ribonuclease A.

The present invention also relates to a method of detecting the single nucleotide polymorphism in the ABCB1 gene, using an antibody, as a mutation of its amino acid sequence resulting from the mutation of the gene. In a case where a mutation is generated in the amino acid sequence of the ABCB1 protein due to a single nucleotide polymorphism of the ABCB1 gene, such a single nucleotide polymorphism can be detected using an antibody against the mutation portion in the amino acid sequence. Since the mutation of the ABCB1 gene utilized in the present invention is a polymorphism which results in a single amino acid mutation in the ABCB1 protein, and as a result, the polymorphism can be detected using an antibody of which the antigen is the mutated portion of the protein, the detection method of the present invention includes the method using such an antibody. Examples of the antibody used in the present invention include polyclonal antibodies and monoclonal antibodies, and their fragments having an antibody-binding activity may be used. Any class of antibodies may also be used, and known special types of antibodies, such as human antibodies, humanized antibodies, and bispecific antibodies, may be used.

A polyclonal antibody may be obtained by immunizing an animal, such as a mouse or a rat, with a peptide containing a substituted portion (in particular, the amino acid portion at position 1251) in the ABCB1 protein, in accordance with a well-known method (Current protocols in Molecular Biology, edit. Ausubel et al. (1987) publish. John Wiley & Sons, Section 11.12-11.13). A monoclonal antibody may be obtained by isolating antibody-producing cells from an animal immunized with the above peptide, fusing the isolated cells with cells such as myeloma cells to prepare hybridoma cells, and producing the monoclonal antibody from the hybridoma cells (Current protocolsin Molecular Biology, edit. Ausubel et al. (1987) publish. John Wiley & Sons, Section 11.411.11). Special antibodies, such as human antibodies (for example, “Functional transplant of megabase human immunoglobulin loci recapitulates human antibody response in mice”, Mendez M. J. et al. (1997) Nat. Genet. 15: 146-156) or humanized antibodies (Methods in Enzymology 203: 99-121 (1991)), may be prepared in accordance with well-known methods.

The present invention also provides a kit for detecting the presence or absence of the single nucleotide polymorphism in single nucleotide polymorphism rs28364274 of the ABCB1 gene in a biological sample. An embodiment is a kit comprising an oligonucleotide (also referred to as a nucleotide probe) which has at least 15 nucleotides in chain length, and hybridizes to a DNA corresponding to a region containing the nucleotide at position 3751 of the CDS of the ABCB1 gene (the nucleotide at position 4169 of the nucleotide sequence of SEQ ID NO: 1).

Another embodiment is a kit comprising oligonucleotides (also referred to as nucleotide primers) capable of amplifying a region containing the nucleotide at position 3751 of the CDS of the ABCB1 gene (the nucleotide at position 4169 of the nucleotide sequence of SEQ ID NO: 1).

Still another embodiment is a kit comprising the nucleotide probe and the nucleotide primers. These kits may be used for an examination using the single nucleotide polymorphism as an index.

These oligonucleotides are ones which specifically hybridize to a DNA containing the ABCB1 gene. The term “to specifically hybridize” as used herein means that a cross-hybridization does not significantly occur with a DNA encoding the other proteins under normal hybridization (selective binding) conditions, preferably under stringent conditions (for example, the conditions described in Sambrook et al., Molecular Cloning, Cold Spring Harbour Laboratory Press, New York, USA, 2nd Ed., 1989). So long as the specific hybridization can be achieved, it is not necessary that the oligonucleotides are completely complementary to the nucleotide sequence of the ABCB1 gene to be detected.

The oligonucleotide of the present invention may be used as a nucleotide probe or nucleotide primers in the method of the present invention. When it is used as primers, the length is generally 15 to 100 bp, and preferably 17 to 30 bp. The primers are not limited, so long as they can amplify at least part of the ABCB1 gene containing the single nucleotide polymorphism (preferably the single nucleotide substitution) portion.

When the oligonucleotide is used as a nucleotide probe, the probe is not limited, so long as it specifically hybridizes to a DNA corresponding a region containing the nucleotide at position 3751 of the CDS of the ABCB1 gene (the nucleotide at position 4169 of the nucleotide sequence of SEQ ID NO: 1). The probe may be a synthetic oligonucleotide, and generally has at least 15 nucleotides in chain length.

The oligonucleotide of the present invention can be prepared, for example, by a commercially available oligonucleotide synthesizer. The probe can also be prepared as a double-stranded DNA fragment obtained by a restriction enzyme treatment or the like.

It is preferable that the oligonucleotide of the present invention is appropriately labeled for the use as a probe. Examples of a labeling method include a labeling method using T4 polynucleotide kinase to phosphorylate the 5′-terminus of the oligonucleotide with ³²P; and a method of introducing substrate nucleotides which are labeled with isotopes such as ³²P, fluorescent dyes, biotin, or the like, using random hexamer oligonucleotides or the like as primers, together with a DNA polymerase such as Klenow enzyme (a random prime method, etc.).

Another embodiment of the detection kit of the present invention is a kit for detecting the presence or absence of the single nucleotide polymorphism on the ABCB1 gene, consisting of a substrate on which a nucleotide probe is immobilized, the probe hybridizing to a DNA corresponding to a region containing the nucleotide at position 3751 of the CDS of the ABCB1 gene (the nucleotide at position 4169 of the nucleotide sequence of SEQ ID NO: 1). This kit may be used for an examination using the single nucleotide polymorphism as an index, and may be prepared in accordance with the above-mentioned method.

Still another embodiment of the detection kit of the present invention is a kit for detecting the presence or absence of the single nucleotide polymorphism on the ABCB1 gene, comprising a forward primer and a reverse primer designed for amplifying a DNA corresponding to a region containing the nucleotide at position 3751 of the CDS of the ABCB1 gene (the nucleotide at position 4169 of the nucleotide sequence of SEQ ID NO: 1). The length of the primers is generally 15 to 100 bp, and preferably 17 to 30 bp. The primers are not limited, so long as it can amplify at least part of the ABCB1 gene containing the single nucleotide polymorphism portion.

For example, sterilized water, physiological saline, vegetable oils, surfactants, lipids, solubilizers, buffers, protein stabilizers (such as BSA and gelatin), preservatives, and the like may be mixed in the kit, if necessary, in addition to the oligonucleotide as the active ingredient. The kit may include an appropriate package and a manual for the present method. The kit may include one or more polymerases, such as a thermostable polymerase, such as tag polymerase.

### EXAMPLES

The present invention now will be further illustrated by, but is by no means limited to, the following Examples.

### Example 1

### Screening for Single Nucleotide Polymorphism Involved in ILD Caused by EGFR Tyrosine Kinase Inhibitors

Case-control association analysis was retrospectively carried out using DNAs prepared from peripheral blood mononuclear cells collected from Japanese patients treated with EGFR tyrosine kinase inhibitors (administration of gefitinib or erlotinib) for advanced non-small cell lung cancer. A patient group included 12 patients who developed acute lung injury after the treatment with EGFR tyrosine kinase inhibitors, and a control group included 56 patients who did not develop acute lung injury after the administration of EGFR tyrosine kinase inhibitors.

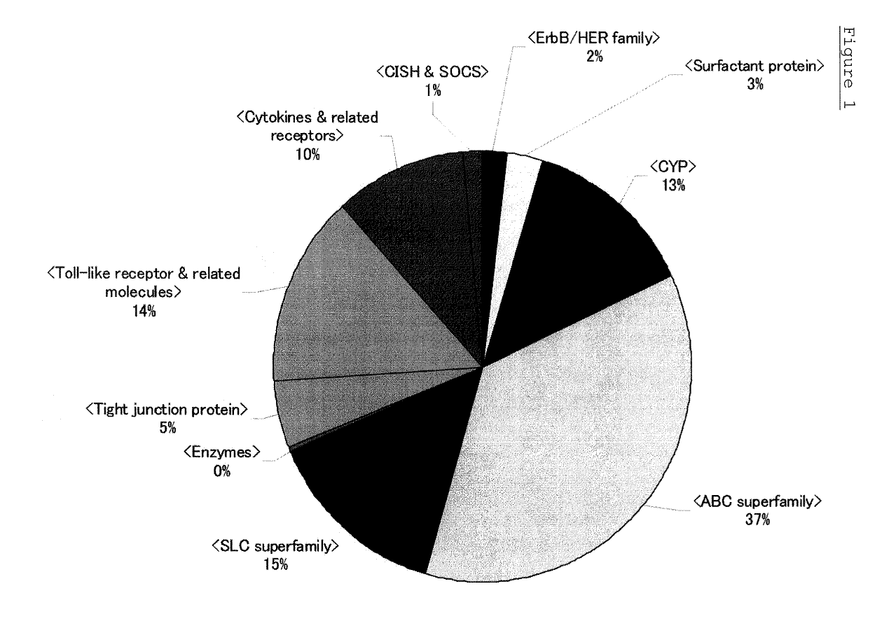

First, taking into consideration mechanisms which might cause drug-induced lung injury, 114 genes were selected as genes which might be involved in acute lung injury, mainly from genes involved in the metabolism of tyrosine kinase inhibitors (CYP, ABC superfamily), genes involved in inflammation (Cytokines & related receptor, Toll-like receptor & related molecules, Tight junction protein), genes which maintained the function of the lung (Surfactant protein), factors for the development of lung cancer (ErbB/HER family), and genes highly expressed in the lung (SLC superfamily). Next, from a large number of SNPs extracted by an SNP search program (Illumina, Inc.) on the basis of gene names, 768 measurable SNPs which result in an amino acid mutation or which locate at a regulation region of gene expression were selected. The percentages of each gene group relative to the total number of SNPs used in this experiment are shown in FIG. 1.

The measurement of 768 SNPs in 114 genes was carried out using Illumina Golden Gate Custom Panel (registered trademark; Illumina, Inc.). The screening conditions were in accordance with the manual attached thereto.

As a result, with respect to single nucleotide polymorphism rs28364274 [a single nucleotide polymorphism of G (wild-type) or A in the nucleotide at position 3751 of the CDS of an ABCB1 gene (the nucleotide at position 4169 of the nucleotide sequence of SEQ ID NO: 1)] of ABCB1 (ATP-binding cassette transporter B1), 4 (33%) of the 12 cases had an A (adenine) allele in the ILD patient group, whereas no case with the A allele was detected in the control group (Table 5). The p value was 0.00016 by Fisher's exact test for the presence of the A allele, and the result indicated that the relationship between the ILD patient group and the presence of the A allele was significant.

TABLE 5

| rs28364274 | AA + AG | GG | Total |
|---|---|---|---|
| ILD | 4 | 8 | 12 |
| Control | 0 | 56 | 56 |
| Total | 4 | 64 | 68 |

### Example 2

### Comparison of Frequency of Mutation in Genotype of rs28364274 in Races

A Reference SNP Cluster Report by NCBI Single Nucleotide Polymorphism (http://www.ncbi.nlm.nih.gov/projects/SNP/) has reported data with the Korean and the Chinese as the Asian sample, and the A allele of rs28364274 was extremely rare in certain non-Japanese races (Table 6).

It was considered from the frequency of occurrence of the A allele of rs28364274 detected in 4 cases of the Japanese patient group described in Example 1 that this A allele was extremely specific to the Japanese, and it was found that this result did not contradict the fact that drug-induced ILD is often developed in the Japanese.

Therefore, it was suggested that the presence of the single nucleotide polymorphism in single nucleotide polymorphism rs28364274 of the ABCB1 gene was involved in the onset of ILD.

TABLE 6

| | | Genotype | | Alleles | |
|---|---|---|---|---|---|
| Race | Samples | A/G | G/G | A | G |
| Subsaharan African | 24 | | 1.000 | | 1.000 |
| Hispanic | 44 | 0.045 | 0.955 | 0.023 | 0.977 |
| European | 44 | | 1.000 | | 1.000 |
| African American | 30 | | 1.000 | | 1.000 |
| Asian | 48 | | 1.000 | | 1.000 |

### Example 3

### Sequencing of ABCB1 Gene

With respect to the probe (SEQ ID NO: 4) used for the measurement of single nucleotide polymorphism rs28364274 of the ABCB1 gene in Example 1, it was again examined whether or not its homologous sequence was present in other genes, and it was found that a highly homologous sequence was present in an ABCB4 gene. This sequence contained single nucleotide polymorphism rs45456698. Therefore, it was further examined whether the SNP detected in Example 1 was single nucleotide polymorphism rs28364274 of the ABCB1 gene.

Primers shown in Table 7 for amplifying the 27th exon region of the ABCB1 gene, the exon region being specific for the ABCB1 gene, were designed on the basis of the genomic sequence of the ABCB1 gene (NCBI, NC 000007.131:c87342564-87132948) and the genomic sequence of the ABCB4 gene (NC_{—}000007.131:c87105019-87031361).

TABLE 7

| | | SEQ | Nucle- |
|---|---|---|---|
| | | ID | otides |
| Primer | Sequence | NO: | (mer) |
| abcb1_27F | 5′-ACCATGCCCGTCCTACTGT-3′ | 5 | 19 |
| (forward | | | |
| primer) | | | |
| abcb1_27R | 5′-CTCTCCACTTGATGATGTCTC | 6 | 25 |
| (reverse | TCAC-3′ | | |
| primer) | | | |

Using the genomic DNA (5 ng) from the 12 patients with acute lung injury, examined in Example 1, the region containing exon 27 of the ABCB1 gene was amplified using the primer set (Table 7; 0.2 pmol/L each) for PCR amplification of exon 27 of the ABCB1 gene, together with Ex-Taq (0.625 units; Takara Shuzo, Kyoto, Japan). In this reaction, the total volume of the reaction liquid was 50 μL, and the PCR conditions were as follows:

a reaction at 94° C. for 5 minutes was carried out;

a cycle consisting of a reaction at 94° C. for 30 seconds, a reaction at 55° C. for 1 minute, and a reaction at 72° C. for 2 minutes was repeated 30 times; and

a reaction at 72° C. for 7 minutes was carried out. To 5 μL of each PCR amplification product, 2 μL of ExoSAP-IT (USB Co., Cleveland, Ohio) was added, and each mixture was treated at 37° C. for 15 minutes, followed by at 80° C. for 15 minutes, and then cooled to 4° C.

The nucleotide sequences of both strands of each product amplified by PCR were directly determined using the primers described in Table 3, together with an ABI BigDye Terminator v1.1 Cycle Sequencing Kit (Life Technologies, Carlsbad, USA). More specifically, 3.2 μL of 1 μmol/L primer, 8 μL of BigDye Terminator v1.1, and 5.8 μL of purified water were added to 3.0 μL of each PCR amplification product so that the total volume became 20 μL, and a cycle consisting of a reaction at 96° C. for 10 seconds, a reaction at 50° C. for 5 seconds, and a reaction at 60° C. for 4 minutes was repeated 25 times, and then each of the resulting mixtures was cooled to 4° C. The excess dye was removed from each mixture, using Centri-Sep™ spin column (Life Technologies), and the total volume (20 μL) of each eluate was dried by a centrifugal evaporator, and subjected to analysis using an ABI Prism 3100 DNA Analyzer (Life Technologies). In this regard, 10 μL of Hi-Di Formamide (Life Technologies) was added to each purified reaction product, and each mixture was subjected to heat shock at 94° C. for 2 minutes.

As a result, the region containing exon 27 of the ABCB1 gene was analyzed, for 12 patients with acute lung injury, to judge the SNP in single nucleotide polymorphism rs28364274 by confirming its nucleotide sequence by waveform. The waveform derived from each individual was output from the ABI Prism 3100 DNA Analyzer, and when only a waveform signal indicating G at position 3751 of the ABCB1 gene was detected, it was judged that the SNP (genotype) was GG. Similarly, when a signal indicating G and a signal indicating A were observed at a ratio of about 1:1, the SNP judgment (genotype) was GA, and when only a signal indicating A was detected, the SNP judgment (genotype) was AA.

As a result, the SNP judgment in 4 samples was GA in 12 samples, and the A allele was detected. The samples in which the A allele was detected in this Example accorded to those in which the A allele was detected by SNP analysis using the Illumina Golden Gate Custom Panel (registered trademark; Illumina, Inc.) in Example 1 (Table 8). It was considered from this result that the SNP with significant difference was located at exon 27 of the ABCB1 gene, and was rs28364274 located at position 3751 of the ABCB1 gene. Valine (V) at position 1251 of the wild-type ABCB1 protein is substituted with isoleucine (I) by the nucleotide substitution of the SNP, and there is a possibility that the drug metabolism in which the ABCB1 gene product is involved will be affected, and it was considered that the single nucleotide polymorphism (mutation) might lead to side effects of drugs.

TABLE 8

| | rs28364274 |
|---|---|
| Sample ID | genotype |
| 1 | GG |
| 2 | GG |
| 3 | GG |
| 4 | GA |
| 5 | GG |
| 6 | GG |
| 7 | GG |
| 8 | GG |
| 9 | GA |
| 10 | GG |
| 11 | GA |
| 12 | GA |

### INDUSTRIAL APPLICABILITY

The present invention can be used for prediction of the risk of developing drug-induced lung injury, for example, in the administration of EGFR tyrosine kinase inhibitors.

Although the present invention has been described with reference to specific embodiments, various changes and modifications obvious to those skilled in the art are possible without departing from the scope of the appended claims.

1. A method of predicting the risk of developing drug-induced lung injury, comprising determining the nucleotide at position 3751 of the CDS of an ABCB1 gene (the nucleotide at position 4169 of the nucleotide sequence of SEQ ID NO: 1).

2. The method according to claim 1, wherein it is judged that the risk of developing drug-induced lung injury is high, when the nucleotide is adenine.

3. A method of detecting the presence or absence of a single nucleotide polymorphism in an ABCB 1 gene in a biological sample, for prediction of the risk of developing drug-induced lung injury, by comparing the ABCB1 gene in the biological sample with a wild-type ABCB 1 gene.

4. The method according to claim 3, wherein the single nucleotide polymorphism is a substitution of adenine for guanine in the nucleotide at position 3751 of the CDS of an ABCB1 gene (the nucleotide at position 4169 of the nucleotide sequence of SEQ ID NO: 1).

5. (canceled)

6. (canceled)

7. The method of detecting the presence or absence of a single nucleotide polymorphism according to claim 3, by detecting the single nucleotide polymorphism in the ABCB1 gene, using an antibody, as a mutation of an amino acid sequence resulting from the single nucleotide polymorphism in the ABCB 1 gene.

8. The method according to claim 1, by detecting the nucleotide at position 3751 of the CDS of the ABCB1 gene, using an antibody, as a mutation of an amino acid sequence resulting from a single nucleotide polymorphism in the nucleotide.

9. An oligonucleotide probe for predicting the risk of developing drug-induced lung injury, which specifically binds to a single nucleotide polymorphism in an ABCB1 gene under selective binding conditions.

10. An oligonucleotide primer for predicting the risk of developing drug-induced lung injury, which amplifies a nucleic acid sequence comprising a single nucleotide polymorphism in an ABCB1 gene.

11. A kit for predicting the risk of developing drug-induced lung injury, comprising the oligonucleotide probe according to claim 9, wherein the presence or absence of the single nucleotide polymorphism in the ABCB 1 gene in a biological sample is detected by comparing the ABCB1 gene in the biological sample with a wild-type ABCB1 gene.

12. The method according to claim 1, said method comprising the steps of:
(a) isolating a DNA comprising a nucleotide site with a single nucleotide polymorphism in an ABCB1 gene from a nucleic acid sample prepared from a subject,
(b) bringing the DNA sample isolated in (a) into contact with a substrate immobilized with an ABCB1 gene fragment comprising the nucleotide site with the single nucleotide polymorphism, as a nucleotide probe, under selective binding conditions, and
(c) detecting the intensity of hybridization of the DNA sample with the nucleotide probe immobilized on the substrate.

13. The method according to claim 3, said method comprising the steps of:
(a) isolating a DNA comprising a nucleotide site with a single nucleotide polymorphism in an ABCB1 gene from a nucleic acid sample prepared from a subject,
(b) bringing the DNA sample isolated in (a) into contact with a substrate immobilized with an ABCB1 gene fragment comprising the nucleotide site with the single nucleotide polymorphism, as a nucleotide probe, under selective binding conditions, and
(c) detecting the intensity of hybridization of the DNA sample with the nucleotide probe immobilized on the substrate.

14. The method according to claim 3, said method comprising the steps of:
(a) isolating a DNA comprising a single nucleotide polymorphism in an ABCB1 gene from a nucleic acid sample prepared from a subject,
(b) determining the nucleotide sequence of the isolated DNA sample, and
(c) comparing the DNA nucleotide sequence determined in (b) with a control.


SEQUENCE LISTING (6 sequences)

SEQ ID NO: 1 - DNA, 4872 bp, Homo sapiens
  CDS (419)..(4261)
tattcagatattctccagattcctaaagattagagatcatttctcattctcctaggagtactcacttcaggaagcaaccagataaaagagaggtgcaacggaagccagaacattcctcctggaaattcaacctgtttcgcagtttctcgaggaatcagcattcagtcaatccgggccgggagcagtcatctgtggtgaggctgattggctgggcaggaacagcgccggggcgtgggctgagcacagccgcttcgctctctttgccacaggaagcctgagctcattcgagtagcggctcttccaagctcaaagaagcagaggccgctgttcgtttcctttaggtctttccactaaagtcggagtatcttcttccaaaatttcacgtcttggtggccgttccaaggagcgcgaggtcggaatggatcttgaaggggaccgcaatggaggagcaaagaagaagaacttttttaaactgaacaataaaagtgaaaaagataagaaggaaaagaaaccaactgtcagtgtattttcaatgtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtgatatcaatgatacagggttcttcatgaatctggaggaagacatgaccaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagatgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaagatactatcttcatttactgataaagaactcttagcgtatgcaaaagctggagcagtagctgaagaggtcttggcagcaattagaactgtgattgcatttggaggacaaaagaaagaacttgaaaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtattcttttctgtattaattggggcttttagtgttggacaggcatctccaagcattgaagcatttgcaaatgcaagaggagcagcttatgaaatcttcaagataattgataataagccaagtattgacagctattcgaagagtgggcacaaaccagataatattaagggaaatttggaattcagaaatgttcacttcagttacccatctcgaaaagaagttaagatcttgaagggtctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggttctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagattgtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtgaactctgactgtatgagatgttaaatactttttaatatttgtttagatatgacatttattcaaagttaaaagcaaacacttacagaattatgaagaggtatctgtttaacatttcctcagtcaagttcagagtcttcagagacttcgtaattaaaggaacagagtgagagacatcatcaagtggagagaaatcatagtttaaactgcattataaattttataacagaattaaagtagattttaaaagataaaatgtgtaattttgtttatattttcccatttggactgtaactgactgccttgctaaaagattatagaagtagcaaaaagtattgaaatgtttgcataaagtgtctataataaaactaaactttcatgtgactggagtcatcttgtccaaactgcctgtgaatatatcttctctcaattggaatattgtagataacttctgctttaaaaaagttttctttaaatatacctactcatttttgtgggaatggttaagcagtttaaataattcctgttgtatatgtctattcacattgggtcttacagaaccatctggcttcattcttcttggacttgatcctgctgattcttgcatttccacat

SEQ ID NO: 2 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 3 - DNA, 511 bp, Homo sapiens
gtgatctgcccgccttggcctcccaaagtgctgggattacaggcgtgagccaccatgcccgtcctactgtggagctttttatggaagaggaattagggaaaagaactattatgagaattaatctatgtgattatggaataggttgtccaagaagccctggacaaagccagagaaggccgcacctgcattgtgattgctcaccgcctgtccaccatccagaatgcagacttaatagtggtgtttcagaatggcagartcaaggagcatggcacgcatcagcagctgctggcacagaaaggcatctatttttcaatggtcagtgtccaggctggaacaaagcgccagtgaactctgactgtatgagatgttaaatactttttaatatttgtttagatatgacatttattcaaagttaaaagcaaacacttacagaattatgaagaggtatctgtttaacatttcctcagtcaagttcagagtcttcagagacttcgtaattaaaggaacagag

SEQ ID NO: 4 - DNA, 121 bp, Homo sapiens
gctcaccgcctgtccaccatccagaatgcagacttaatagtggtgtttcagaatggcagartcaaggagcatggcacgcatcagcagctgctggcacagaaaggcatctatttttcaatgg

SEQ ID NO: 5 - DNA, 19 bp, Homo sapiens
accatgcccgtcctactgt

SEQ ID NO: 6 - DNA, 25 bp, Homo sapiens
ctctccacttgatgatgtctctcac